(19) United States
(12) Patent Application Publication
(10) Pub. No.: US 2010/0004491 A1
(43) Pub. Date: Jan. 7, 2010

(54) Method Of Fuse Oil Decanting In An Ethanol Plant

(75) Inventor: D. Christopher Romer, Memphis, TN (US)

Correspondence Address: AIR LIQUIDE;Intellectual Property, 2700 POST OAK BOULEVARD, SUITE 1800, HOUSTON, TX, 77056, US

(73) Assignee: Lurgi PSI, Inc., Memphis, TN (US)

(21) Appl. No.: 12/494,689

(22) Filed: Jun. 30, 2009

Related U.S. Application Data
(60) Provisional application No. 61/078,024, filed on Jul. 3, 2008.

(51) Int. Cl. C07C 29/74; B01D 3/32

(52) U.S. Cl. 568/916; 202/234

(57) ABSTRACT

An apparatus including an ethanol stream, wherein said ethanol stream contains greater than 5% of water; a dehydration means, wherein the water content of said ethanol stream is reduced to less than 5%; a desiccation means, wherein the water content of said ethanol stream is reduced to less than about 1%; a 200 proof receiver, wherein said 200 proof receiver comprises a sample port or a sensor to allow the percentage of water in the ethanol to be determined, and wherein said 200 proof receiver comprises an outlet; a heat exchanger to heat said ethanol stream with less than 1% water, and a mixing manifold downstream of said heat exchanger, wherein said heated ethanol stream with less than 1% water is combined with fusal oils from a fusal oil decanter is provided.

This application claims the benefit of U.S. Provisional Application No. 61/078,024, filed Jul. 3, 2008, the entire contents of which are incorporated herein by reference.

### BACKGROUND

A variety of cereal grains and other plants are grown for use as food. Major cereal grains include corn, rice, wheat, barley, sorghum (milo), millets, oats, and rye. Other plants include potatoes, cassava, and artichokes. Corn is the most important cereal grain grown in the United States. A mature corn plant consists of a stalk with an ear of corn encased within a husk. The ear of corn consists of about 800 kernels on a cylindrical cob. The kernels are eaten whole and are also processed into a wide variety of food and industrial products. The other parts of the corn plant (i.e., the stalk, leaves, husk, and cob) are commonly used for animal feed, but are sometimes processed into a variety of food and industrial products.

In more detail, the corn kernel consist of three main parts: (1) the pericarp; (2) the endosperm; and (3) the germ. The pericarp (also known as the seed coat or bran) is the outer covering of the kernel. It consists primarily of relatively coarse fiber. The endosperm is the energy reserve for the plant. It consists primarily of starch, protein (also known as gluten), and small amounts of relatively fine fiber. The germ (also known as the embryo) consists primarily of oil and a miniature plant with a root-like portion and several embryonic leaves.

Starch is stored in a corn kernel in the form of discrete crystalline bodies known as granules. Starch is a member of the general class of carbohydrates known as polysaccharides. Polysaccharides contain multiple saccharide units (in contrast to disaccharides which contain two saccharide units and monosaccharides which contain a single saccharide unit). The length of a saccharide chain (the number of saccharide units in it) is sometimes described by stating its “degree of polymerization” (abbreviated to D.P.). Starch has a D.P. of 1000 or more. Glucose (also known as dextrose) is a monosaccharide (its D.P. is 1). Saccharides having a D.P. of about 5 or less are sometimes referred to as sugars.

As mentioned above, the pericarp and endosperm of the corn kernel contain fiber. The fiber comprises cellulose, hemicellulose, lignin, pectin, and relatively small amounts of other materials. Fiber is present in relatively small amounts in the corn kernel, but is present in much greater amounts in other corn components such as the cob, husk, leaves, and stalk. Fiber is also present in other plants. The combination of cellulose and lignin is sometimes known as lignocellulose and the combination of cellulose, lignin, and hemicellulose is sometimes known as lignocellulosic biomass. As used herein, the term “fiber” (and its alternative spelling “fibre”) refers to cellulose, hemicellulose, lignin, and pectin.

A wide variety of processes have been used to separate the various components of corn. These separation processes are commonly known as corn refining. One of the processes is known as the dry milling process. In this process, the corn kernels are first cleaned and then soaked in water to increase their moisture content. The softened corn kernels are then ground in coarse mills to break the kernel into three basic types of pieces—pericarp, germ, and endosperm. The pieces are then screened to separate the relatively small pericarp and germ from the relatively large endosperm. The pericarp and the germ are then separated from each other. The germs are then dried and the oil is removed. The remaining germ is typically used for animal feed. The endosperm (containing most of the starch and protein from the kernel) is further processed in various ways. As described below, one of the ways is to convert the starch to glucose and then ferment the glucose to ethanol.

Fermentation is a process by which microorganisms such as yeast digest sugars to produce ethanol and carbon dioxide. Yeast reproduce aerobically (oxygen is required) but can conduct fermentation anaerobically (without oxygen). The fermented mixture (commonly known as the beer mash) is then distilled to recover the ethanol. Distillation is a process in which a liquid mixture is heated to vaporize the components having the highest vapor pressures (lowest boiling points). The vapors are then condensed to produce a liquid that is enriched in the more volatile compounds.

With the ever-increasing depletion of economically recoverable petroleum reserves, the production of ethanol from vegetative sources as a partial or complete replacement for conventional fossil-based liquid fuels becomes more attractive. In some areas, the economic and technical feasibility of using a 90% unleaded gasoline-10% anhydrous ethanol blend (“gasohol”) has shown encouraging results. According to a recent study, gasohol powered automobiles have averaged a 5% reduction in fuel compared to unleaded gasoline powered vehicles and have emitted one-third less carbon monoxide than the latter. In addition to offering promise as a practical and efficient fuel, biomass-derived ethanol in large quantities and at a competitive price has the potential in some areas for replacing certain petroleum-based chemical feedstocks. Thus, for example, ethanol can be catalytically dehydrated to ethylene, one of the most important of all chemical raw materials both in terms of quantity and versatility.

### SUMMARY

The present invention is an apparatus including an ethanol stream, wherein said ethanol stream contains greater than 5% of water; a dehydration means, wherein the water content of said ethanol stream is reduced to less than 5%; a desiccation means, wherein the water content of said ethanol stream is reduced to less than about 1%; a 200 proof receiver, wherein said 200 proof receiver comprises a sample port or a sensor to allow the percentage of water in the ethanol to be determined, and wherein said 200 proof receiver comprises an outlet; a heat exchanger to heat said ethanol stream with less than 1% water, and a mixing manifold downstream of said heat exchanger, wherein said heated ethanol stream with less than 1% water is combined with fusal oils from a fusal oil decanter.

### DESCRIPTION OF PREFERRED EMBODIMENTS

Rerouting of fusel oil line from 200 proof receiver to downstream of product cooler and sample point. This change allows the sampling of 200 proof without fusels present, and gives a better indication of how well the mole sieves are operating.

A typical design for the drying and distillation area may connect the fusel oil decanter discharge into the 200 proof receiver, thereby mixing the fusel oil into the dehydrated ethanol (200 proof). While mixing these two may be an ultimate goal, depending on the overall system design, there is a problem with this design decision, and a little background will make this clearer.

One of the main goals of the plant is to produce ethanol so that when blended with gasoline, it will meet the specifications required by the ASTM specifications for fuel ethanol, and perhaps even more stringent requirements of the purchaser. One of the key factors is the amount of water that is allowed in the ethanol. The ASTM spec requires no more that 1% by volume—some purchasers may want a smaller number. The fusel oils are mixture of organic liquids with some water, which must be removed from one of the distillation columns and can be added to the 200-proof ethanol for sale.

Typically, the last major process step for the ethanol prior to entering the 200-proof receiver is to pass through molecular sieves as a gas where the water entrained with ethanol is captured and the ethanol passes through. In a typical drying and distillation system, this dehydrated, gaseous ethanol is then condensed to form a liquid under pressure and placed in the 200-proof ethanol receiver.

The key issue is that the ethanol is only a liquid because it is under pressure. As no provision is typically made to cool this stream to ambient conditions, a sample of this liquid cannot be taken from this tank. In addition, because both the ethanol and the fusel oils can, and will, contain some water, by mixing them in this tank, it is impossible to directly sample each flow and determine which flow is contributing what amount of water.

In one embodiment of the first inventive design, the fusel oils are being moved to down stream of the heat exchanger and a sample port and before the flowmeter for the plant productions rate.

In one embodiment of the second improvement the Temperature compensation has been further improved by relocating it downstream of the combined flow.

In one embodiment of the third inventive design, the heat exchanger may be upstream of the receiver to cool the product before it is received. This would allow the sampling on the suction side of a pump which is preferable from a control stand point. The heat exchanger would have to be able to take a the higher fluctuating loads and would therefore be somewhat bigger than the current design.

What is claimed is:

1. An apparatus comprising;
an ethanol stream, wherein said ethanol stream contains greater than 5% of water,
a dehydration means, wherein the water content of said ethanol stream is reduced to less than 5%,
a desiccation means, wherein the water content of said ethanol stream is reduced to less than about 1%,
a 200 proof receiver, wherein said 200 proof receiver comprises a sample port or a sensor to allow the percentage of water in the ethanol to be determined, and wherein said 200 proof receiver comprises an outlet,
a heat exchanger to heat said ethanol stream with less than 1% water, and
a mixing manifold downstream of said heat exchanger, wherein said heated ethanol stream with less than 1% water is combined with fusal oils from a fusal oil decanter.

2. The apparatus of claim 1, wherein said desiccation means comprises a molecular sieve.

3. The apparatus of claim 1, further comprising a flow transmitter downstream of the mixing manifold and a temperature transmitter upstream of the mixing manifold.

4. The apparatus of claim 1, further comprising a flow transmitter and a temperature transmitter downstream of the mixing manifold.

5. The apparatus of claim 1, further comprising a flow transmitter downstream of the mixing manifold, and a temperature transmitter downstream of said heat exchanger and upstream of said 200 proof receiver.

6. A method comprising;
providing an ethanol stream, wherein said ethanol stream contains greater than 5% of water,
providing a dehydration means, wherein the water content of said ethanol stream is reduced to less than 5%,
providing a desiccation means, wherein the water content of said ethanol stream is reduced to less than about 1%,
providing a 200 proof receiver, wherein said 200 proof receiver comprises a sample port or a sensor to allow the percentage of water in the ethanol to be determined, and wherein said 200 proof receiver comprises an outlet,
providing a heat exchanger to heat said ethanol stream with less than 1% water, and
providing a mixing manifold downstream of said heat exchanger, wherein said heated ethanol stream with less than 1% water is combined with fusal oils from a fusal oil decanter.
